SEQUENCE LISTING (61 sequences)

SEQ ID NO: 1 - DNA, 101 bp, Homo sapiens
gatattccatgtggtgtggggctggagaagcctgggcgccactggtggaggcagcccgaggcaggcacaacataaccgaggacagcaagactccctcagaa

SEQ ID NO: 2 - DNA, 23 bp, Homo sapiens
aaataacatttaataggacaagg

SEQ ID NO: 3 - DNA, 118 bp, Homo sapiens
aatgaatgtctctaccccaaactatgagagctccaggggcagagacttgttattttgtccattcttatccccagagtttggcaactggtcagcacttgataaataaaggaaagaatgt

SEQ ID NO: 4 - DNA, 32 bp, Homo sapiens
gctgaccagttgccaaactctggggataagaa

SEQ ID NO: 5 - DNA, 104 bp, Homo sapiens
ctcgggcacagcattcatggaaaggaaaggtgtacgggacatgcccgaggatcctcaagtcccacagaaacagggaggggctggggaagctcattctacagatg

SEQ ID NO: 6 - DNA, 26 bp, Homo sapiens
aggtgtacgggacatgcccgaggatc

SEQ ID NO: 7 - DNA, 104 bp, Homo sapiens; identical to SEQ ID NO: 5

SEQ ID NO: 8 - DNA, 26 bp, Homo sapiens
aggtgtacgggacatgcccgaggatc

SEQ ID NO: 9 - DNA, 102 bp, Homo sapiens
tggggtttaatagatgaattttatcaggttgaggaaattttatttctaatctgctcagtgttttttcatcacaagagtgttggattttgttaatatttttgt

SEQ ID NO: 10 - skipped

SEQ ID NO: 11 - DNA, 106 bp, Homo sapiens
tcagtctcccaattattgctaattgatggaagaagaagaccgagtgtggtcttcctttttaaaaagctacctccgttctcgcgccattgcactccagctgggcgac

SEQ ID NO: 12 - DNA, 23 bp, Homo sapiens
tttaaaaaggaagacacactcgg

SEQ ID NO: 13 - DNA, 106 bp, Homo sapiens; identical to SEQ ID NO: 11

SEQ ID NO: 14 - DNA, 22 bp, Homo sapiens
tttaaaaaggaagacacactcg

SEQ ID NO: 15 - DNA, 114 bp, Homo sapiens
cctttgcttatttagcttctaatccattaatcatggagagcttcattttttggggggggggggggggcgggggacttctcagacctcatcaaactctactctaaagcactacca

SEQ ID NO: 16 - DNA, 23 bp, Homo sapiens
tcatggagagcttcattttttgg

SEQ ID NO: 17 - DNA, 23 bp, Homo sapiens
ggagagcttcattttttgggggg

SEQ ID NO: 18 - DNA, 23 bp, Homo sapiens
catggagagcttcattttttggg

SEQ ID NO: 19 - DNA, 105 bp, Homo sapiens
gcgcaccgccctctcctcccctcggccgcagtccccgcgcgccccgaggcgtgcttgccctccgccaagcgcgcccactaccctgcccgctcctgcagggggcta

SEQ ID NO: 20 - DNA, 23 bp, Homo sapiens
ggcggagggcagcacgctcgggg

SEQ ID NO: 21 - DNA, 23 bp, Homo sapiens
tggcggagggcagcacgctcggg

SEQ ID NO: 22 - DNA, 107 bp, Homo sapiens
ctcgtgggaacctcttattctgtttatgactcctcagcggtgcagaaagttattccttcccttgctggacaccacatcaaaggaggcccacaggtgtttcttccttc

SEQ ID NO: 23 - DNA, 28 bp, Homo sapiens
atgtggtgtccagcaagggaaggaataa

SEQ ID NO: 24 - DNA, 111 bp, Homo sapiens
ccagagagcaaggccctggccaggaaggtgtcctgcaagctgtcgctgcgccagcccggggaggtgagtgtgtgggctgggcagtccttatggtcatgctaagctggaggc

SEQ ID NO: 25 - DNA, 23 bp, Homo sapiens
aagctgtcgctgcgccagcccgg

SEQ ID NO: 26 - DNA, 23 bp, Homo sapiens
gctgtcgctgcgccagcccgggg

SEQ ID NO: 27 - DNA, 23 bp, Homo sapiens
agctgtcgctgcgccagcccggg

SEQ ID NO: 28 - DNA, 96 bp, Homo sapiens
agaaagcatggtacagtggggaaggtacatggatggatgaagatggccatgacaatgagattaagagacaccatggtgagagagcttcacaaagcc

SEQ ID NO: 29 - DNA, 23 bp, Homo sapiens
gtctcttaatctcattgtcatgg

SEQ ID NO: 30 - DNA, 89 bp, Homo sapiens
ttcttcattgggccgaactttctggtcctcatccaacagctcttctatcacgtgtcgaaagtgtcagccaatgatgtcaagcctcttga

SEQ ID NO: 31 - DNA, 23 bp, Homo sapiens
acacgtgatagaagagctgttgg

SEQ ID NO: 32 - DNA, 106 bp, Homo sapiens
acttagagtaggcatggcagagggagctaacccataggaaattaaggaatttgaagaatctttgaaagttcatctcatttaacttctttaaaacaaaatttgtgaa

SEQ ID NO: 33 - DNA, 28 bp, Homo sapiens
agctaacccataggaaattaaggaattt

SEQ ID NO: 34 - DNA, 106 bp, Homo sapiens; identical to SEQ ID NO: 32

SEQ ID NO: 35 - DNA, 28 bp, Homo sapiens
agctaacccataggaaattaaggaattt

SEQ ID NO: 36 - DNA, 105 bp, Homo sapiens
ccgcccagcctcaggtttcagatctgagttggtcactccttcacttggaacccactcttgtgtggcctccgtgttcaggctgctgggtggggccggccaggctgt

SEQ ID NO: 37 - DNA, 23 bp, Homo sapiens
acttggaacccactcttgtgtgg

SEQ ID NO: 38 - DNA, 23 bp, Homo sapiens
cacggaggccacacaagagtggg

SEQ ID NO: 39 - DNA, 105 bp, Homo sapiens; identical to SEQ ID NO: 36

SEQ ID NO: 40 - DNA, 23 bp, Homo sapiens
acttggaacccactcttgtgtgg

SEQ ID NO: 41 - DNA, 23 bp, Homo sapiens
cacggaggccacacaagagtggg

SEQ ID NO: 42 - DNA, 106 bp, Homo sapiens
gcttggcagctgctaaggcctcggcccaggcctgaagaggctgcccccacaccgctggttcatggttcctggccctccgtggcttgtctctgtccactcttgtgcc

SEQ ID NO: 43 - DNA, 23 bp, Homo sapiens
gaaccatgaaccagcgcgtgtgg

SEQ ID NO: 44 - DNA, 23 bp, Homo sapiens
accatgaaccagcgcgtgtgggg

SEQ ID NO: 45 - DNA, 23 bp, Homo sapiens
ccatgaaccagcgcgtgtggggg

SEQ ID NO: 46 - DNA, 23 bp, Homo sapiens
aaccatgaaccagcgcgtgtggg

SEQ ID NO: 47 - DNA, 106 bp, Homo sapiens; identical to SEQ ID NO: 42

SEQ ID NO: 48 - DNA, 23 bp, Homo sapiens
gaaccatgaaccagcgcgtgtgg

SEQ ID NO: 49 - DNA, 23 bp, Homo sapiens
accatgaaccagcgcgtgtgggg

SEQ ID NO: 50 - DNA, 23 bp, Homo sapiens
ccatgaaccagcgcgtgtggggg

SEQ ID NO: 51 - DNA, 23 bp, Homo sapiens
aaccatgaaccagcgcgtgtggg

SEQ ID NO: 52 - DNA, 105 bp, Homo sapiens
acaacgatgtgtataaatctccaaaggcatcatgctaagtgacaggagtcagtctcaaaaagttacataccgtgtgattccattgatatgacatcctctaaaaga

SEQ ID NO: 53 - DNA, 28 bp, Homo sapiens
aaggcatcatgctaagtgacaggagtca

SEQ ID NO: 54 - DNA, 105 bp, Homo sapiens; identical to SEQ ID NO: 52

SEQ ID NO: 55 - DNA, 28 bp, Homo sapiens
aaggcatcatgctaagtgacaggagtca

SEQ ID NO: 56 - DNA, 106 bp, Homo sapiens
cagttaagtgggagaaaaaataaaagtaaataacatttaataggacaaggatagtaagtcagagtgtggacttttcattgactcacatgaggtctctgtttcaact

SEQ ID NO: 57 - DNA, 23 bp, Homo sapiens
aaataacatttaataggacaagg

SEQ ID NO: 58 - DNA, 106 bp, Homo sapiens; identical to SEQ ID NO: 56

SEQ ID NO: 59 - DNA, 23 bp, Homo sapiens
aaataacatttaataggacaagg

SEQ ID NO: 60 - DNA, 108 bp, Homo sapiens
tcaaccccgactgtgccgccatcacccagcggatggtggtggatttcgctggcgtgaaggacaaggtgtgcatgcctgacccgttgtcagacctggaaaaagggccgg

SEQ ID NO: 61 - DNA, 23 bp, Homo sapiens
tttcgctggcatgaaggacaagg

(19) United States
(12) Patent Application Publication
(10) Pub. No.: US 2024/0011053 A1
(43) Pub. Date: Jan. 11, 2024

(54) Method of early diagnosis and composition of gRNA for treating mental health diseases using genome editing and cell and gene therapy

(71) Applicant: Mohammad A Heidaran, Potomac, MD (US)

(72) Inventor: Mohammad A Heidaran, Potomac, MD (US)

(21) Appl. No.: 17/832,572

(22) Filed: Jun. 13, 2022

(51) Int. Cl. C12N 15/90; C12N 9/22; C12N 15/86; A61K 9/127

(52) U.S. Cl. CPC C12N 15/907; C12N 9/22; C12N 2310/20; A61K 9/127; C12N 15/86

(57) ABSTRACT

Mental diseases include a heterogenous array of diseases that affect brain function. Evidence suggests that some of more serious conditions that lead to suicide may have some genetic and epigenetic determinants. Single Nucleotide Polymorphism (SNPs) have been identified that correlate with a variety of mental conditions and diseases. In this patent application we describe a method by which subjects with life threatening mental health disease can be identified using Single Nucleotide Polymorphism (SNP) analysis and such associated SNPs is corrected using in vivo genome editing technologies.

### BACKGROUND

Genome editing technology utilizes a sequence-specific DNA binding RNA (gRNA) which targets the mutation, and the correction of nucleic acid is performed with help from different forms of endonucleases including meganucleases, zinc-finger nucleases (ZFNs), and transcription activator-like effector nucleases (TALENs), The clustered regularly interspaced short palindromic repeats (CRISPR)-Cas9 (CRISPR associated protein 9) as a programmable nuclease. In recent years inactive nuclease are also used which sometime referred to as base editing and prime editing (FIG. 1).

Genome editing has already been used to correct single nucleotide mutations and treatment of monogenetic diseases. These corrections of target gene can be performed using ex vivo editing of cells that have mutant gene or alternatively the gene editing materials can be delivered to target organs in vivo. This method commonly referred to as in vivo gene editing. The major issue with genome editing is the extent to which gRNA is specific to target site in genome and depending on the nuclease activity if there is a chance for alteration in genome as defined as off target edit or other genomic aberrations like translocation. Accordingly, the most aspect of developing a therapy which is safe and effective is to design the gRNA with minimal off target effects based on computational biological tools described here and to select the gRNA and nuclease complex with minimal off target effects in vivo.

FIG. 1A, Mechanism by which a complex between nuclease and guide RNA can be used to change DNA.

### Genome Editing Delivery

For example as performed herein genome editing is done by use of CRISPR-Cas9 and guide RNA (gRNA) genome editing complex. The gRNA is synthesized chemically and delivered in vivo in complex with RNA that encode for CAS9 nuclease. To assist uptake of this complex the gRNA and RNA encoding nuclease is mixed with lipids and cholesterol to manufacture Lipid nanoparticles (LNP) that contain gRNA and RNA encoding for the nuclease. For RNP, Cas9 protein (160 kDa) is usually combined with gRNA (34 kDa) to form an RNP complex before introducing it into a cell. Since the RNP complex is ready for action, the RNP complex is delivered to the nucleus via nuclear localization signal (NLS) and performs genome editing once it enters the cells. In other embodiments this complex can be delivered using viruses or different method of delivery of endonuclease one of the three forms; DNA, RNA, or protein (FIG. 2).

FIG. 2. Various methods for delivering CRISPR-Cas9 components.

Schematic illustrations represent varieties of the tools to deliver genome editing components. The delivery tools are roughly classified into three categories: biological, using viruses to deliver gene editing reagents. These viruses include AdV, AAV, Retroviruses or Lentiviruses and Virus like particles. The chemical method which is described here involves making a lipid nanoparticle which is loaded with components of gene editing reagents that include gRNA and Cas9 endonuclease protein or mRNA.

### SUMMARY OF INVENTION

In this invention we teach how mental health diseases associated with certain SNPs identified in Excel document I and Table I can be cured by change of variant form SNP to reference nucleotide when such variant is identified in patient. The reference nucleotide for each SNP is identified in Excel document I. The method of treatment involves the following steps:
1) Identify if variant forms are present in patient by performing diagnostic test that detect such SNPs. This can be done by performing qPCR analysis to detect the variant listed in Excel document 1.
2) Patients with one of more of variants are administer a drug product that preferably consists of mRNA encoding CAS9 endonuclease plus gRNA and Specific lipids (method by which the DP is manufactured and formulated is described in this invention).
3) The drug off target effects are minimized by direct administration of the drug product into either different regions of brain or into cerebrospinal fluid and by designing gRNA with high specificity.

Foe example, delivery of DP into cerebrospinal fluid can be done by conventional injection syringe. For direct delivery of DP into different region of brain a device manufactured by clear point will be used. The delivery device is described herein and further illustrated in FIG. 6 of this application.

The overall objective of this patent to describe steps needed to enable modification of specific target SNP using nonviral LNP-mediated CRISPR-Cas9 mRNA-based strategy for in vivo brain-specific genome editing. We accomplish by identifying gRNA, mRNA encoding Cas9 endonuclease and Lipid Nanoprticle LNP formulation to maximize the in vivo mRNA delivery efficacy.

All sgRNAs used in this patent sourced from Synthego using their “end-modified” synthesis. Briefly, the first and last three bases of these gRNA were synthesized using 2′-O-Methyl-nucleosides and joined via 3′ phosphorothioate bonds. All other bases throughout the rest of the molecule are traditional ribonucleosides and phosphate bonds that are typical for native RNA.

Currently there are no curative option for mental diseases. The use of genome editing to treat sever mental health diseases is nonobvious and has not been described elsewhere. In recent few years several SNPs have been associated with severe mental health disease (see reference section herein). Furthermore, SNPs with positive correlation with severe mental health outcome including suicide are being identified in region of human genome which are intergenic, or located in the intron non coding region of certain gene. It is postulated but unexpected that changes in certain target SNPs in non coding intron regions or intergenic sites will affect global expression of genes which are important for maintenance of normal mental health.

Mental disorders are treated with anti-anxiety medicine, anti-depressants, and behavioral modifications using therapies. The concept of treating mental disease at genetic levels is not currently even speculated as current treatments involves modifying the symptoms using anti-depressant or anti-anxiety medicine.

The method described here allows for genetic and epigenetic testing of the patient and based on SNPs and other target identified patient in high risk of death can be treated with cell and gene therapies.

The products described herein include targeted genome editing drug products that takes advantage of known technologies to edit patient genome allowing specific SNPs in patient to be corrected. In another embodiment it is also practical to introduce a functioning copy of the gene malfunctioning to patient via several delivery mechanisms including Adeno Associated Viruses (AAV) or lentiviral vectors for example.

In another embodiment describe in this application the expression of mRNA and proteins regulating optimal mental health can be modulated by in vivo genome editing techniques as described here. The putative target sequence can be the following: 1) noncoding region of certain genes silenced in individuals suffering from serious mental health disease; 2) specific sequences of the promoter and enhancer regions of specific genes; 3) sequences in the coding region of specific genes that contribute to destabilization of mRNA half-life. Furthermore, it is also envisioned that protein expression can be modulated by introducing direct sequence specific edit in the mRNA or SiRNA. Target genes that are implicated in serious and life-threatening conditions are summarized in Excel document I and Table I below.

| | | | | | | | |
|---|---|---|---|---|---|---|---|
| | | | | | | | Fibroblast |
| Brain | Glial | | | | Insulin | | Growth |
| Derived | Derived | Neurotrophic | Vascular | Platelet | like | Epidermal | Factor |
| Neurotrophic | Neurotrophic | Growth | Endothelial | Derived | Growth | Growth | (FGF) |
| Factor | Factor | Factor | Growth | Growth | Factor | Factor | basic and |
| (BDNF) | (GDNF) | (NGF) | Factor | Factor A | (IGF-I) | (EGF) | acidic |

| | | | | | |
|---|---|---|---|---|---|
| Hepatocyte | Tyrosine | Aromatic | GTP | Tryptophan | Insulin |
| Growth | Hydroxylase | amino | cyclohydrolase | Hydroxylation | |
| Factor | (TH) | acid | I (GTPCHI) | (Tph) | |
| (HGF) | | Decarboxylase | | | |
| | | (AADC) | | | |

In vivo genome editing of SNPs for mental health diseases:

gRNA sequence targeting SNPs identified in Excel document I was designed by SNP-CRISPR a web-based tool for SNP specific genome editing (PMM:31822517, PMC7003079, Chen C L et al., 2020). The gRNA was designed based on SNP identified in excel document I and Table I and gene edit conversion to the reference nucleotide. The list of gRNAs for gene edit of each target SNP is summarized in excel document I. The Sequence ID for target genes and sequence for gRNA is provided in an excel document II.

The excel document I contains the following categories of information for each target gene
Column 1: Identifier for each SNP targeted
Column 2: The identifier for chromosome harboring the target SNP
Column 3: The exact location of the target gene
Column 4: The exact strand of DNA (−) versus (+)
Column 5: The identity of nucleotide (AGCT) which is expressed in healthy individuals referred to as reference sequence
Column 6: The sequence of polymorphism found in patients with severe mental health disease
Column 7: The sequence surrounding the nucleotide mutated in individuals with severe mental health disease
Column 8: gRNA sequence
Column 9-11: Measure of quality of representative gRNA specificity and off target probability
Column 12: Location of edit for example in exon, intron or intergenic regions.

The excel document II includes the following columns:
Column 1: SEQID of target genes SNP
Column 2: Sequence information providing at least 30 nucleotides flanking the 3 and 5′ or gRNA sequence forward or reverse

This is a reference to ASC II test file which was added in response to missing part that contains SEQID for all sequences provided in this patent application

The file name is SequenceID_Patent(1).text corresponds to The excel document II

The size is 3.96 KB, 4KB on disk

Date created Jun. 20, 2022 at 10:59 AM

The file name is Target_Gene_SNP_Corijan corresponds to the excel document I

The size is 4.96 KB, 9.00 KB on disk

Date created Jun. 20, 2022, 11:04 AM

For in vivo gene therapy, Cas9 mRNA and targeted sgRNA are coloaded with LNP at a dose of 1.0, 2.0, and 3.0 mg/kg in total RNA. The optimal dose for treatment is 0.05, 0.5, 1 mg/Kg of LNP formulated with mRNA for Cas9 and gRNA used at equal molar ratio.

### Mechanism of Action

It has been demonstrated in vitro and in vivo when lipid nanoparticles are in complex with gRNA and Cas9 mRNA, this LNP particles are taken up by the cell in vivo when injected locally or intravenously. In the present invention it is expected that neuronal cells in brain which have specific SNPs to take up RNP in complex with gRNA and Cas9 mRNA. Upon entry to neuronal cells the cells will translate mRNA into Cas9 protein endonuclease which becomes directed to the target SNPs based on the homology of gRNA directed to a specific SNP. The gRNA is designed to be in complex with Cas9 protein. Once the nuclease, Cas9 protein comes in close proximity of the SNP it makes a double stranded break in target DNA resulting in a subsequent repair that changes the observed SNP to the reference nucleotide sequence. The efficiency of this mutation is largely dependent on the specificity and sequence of gRNA and activity of Cas9 endonuclease. It is also expected that LNP complex to have some off target effects which is defined as changes in DNA outside of target SNP which may or may not be consequential. The off target for LNP constructed will be minimized based on local delivery into brain. The LNP complex will remain in brain microenvironment and cannot across blood brain barrier thus the systemic effects or off target effects is not observed. In addition, the gRNA is designed to minimize any off target effects in cells other than neurons in brain (Refer to FIG. 3).

FIG. 3: Insert Schematic showing LNP delivery into brain and uptake by neuronal cells and edit of target SNP.

### Materials and Methods

### General Reagents

All reagents and chemicals purchased from Sigma-Aldrich unless otherwise specified. 2′ O-methyl and phosphorothioate end-modified sgRNA and template DNA sequences were acquired from Sigma-Aldrich and stored in RNAse-free Tris EDTA-buffer

pH 7.0 (Thermo Fisher). C12-200 lipid [25] was acquired from CordonPharma (Plankstadt, Germany), DOPE from Lipoid (Steinhausen, Switzer-land), Cholesterol and PEG-DMG from Sigma-Aldrich (Zwijndrecht, the Netherlands) and DOTAP from Merck (Darmstadt, Germany).

### Algorithm for Guide RNA (gRNA) Design and Synthesis

gRNA sequence targeting SNPs identified in Excel document I was designed by SNP-CRISPR a web-based tool for SNP specific genome editing (PMID:31822517, PMC7003079, Chen C L et al., 2020).

In some cases sgRNA was designed using <http://crispor.tefor.net/>. The sgRNA included in this patent application have determined in silico to have minimal off target effects with maximal editing efficiency.

The gRNA matrix or backbone is also described herein as an example.

5′!NNNNN NNNNN NNNNN NNNNN NGG!3′

STEP 1: Find all 23 bp genomic sites of the form 5′-N 20 NGG-3′ near your intended target site (ideally ±50 bp). These may reside on the + or −strand.

TGTACAAAAAAGCAGGCTTTAAAGGAA
CCAATTCAGTCGACTGGATCCGGTACC
AAGGTCGGGCAGGAAGAGGGCCTATTT
CCCATGATTCCTTCATATTTGCATATA
CGATACAAGGCTGTTAGAGAGATAATT
AGAATTAATTTGACTGTAAACACAAAG
ATATTAGTACAAAATACGTGACGTAGA
AAGTAATAATTTCTTGGGTAGTTTGCA
GTTTTAAAATTATGTTTTAAAATGGAC
TATCATATGCTTACCGTAACTTGAAAG
TATTTCGATTTCTTGGCTTTATATATC
TTGTGGAAAGGACGAAACACCGNNNNN
NNNNNNNNNNNNNNGTTTTAGAGCTAG
AAATAGCAAGTTAAAATAAGGCTAGTC
CGTTATCAACTTGAAAAAGTGGCACCG
AGTCGGTGCTTTTTTTCTAGACCCAGC
TTTCTTGTACAAAGTTGGCATTA
PAM
GUUUUAGAGCUA
UAAAAUU CGAU
GAA
GA
A
A
. . .

matches
target sequence
Guide RNA
gRNA
scaffold

GNNNNNNNNNNNNNNNNNNN5′-
AGCCACG
UCGGUGC
G
AAGGCUAGUCCGUUAUCAAGUGAAAAAGUUC
3′-UUUU

STEP 2: Using NCBI blast, select sequences for which none or very few sequences of the form 5′-NNNNN NNBBB BBBBB BBBBB NGG-3′ exist at any other location in the human genome (here the B's represent the actual bases at the target genomic location).

STEP 3: Incorporate 19 bp of the selected target sequence as highlighted here: 5′-NNNNN NNNNN NGG-3′ into the DNA fragment as indicated below:

Step 4: This 455 bp fragment bears all components necessary for gRNA expression, namely: U6 promoter+target sequence+guide RNA scaffold+termination signal. Synthesize this as a gBlock from IDT (http://www.idtdna.com/pages/products/genes/gblocks-gene-fragments)

TargetU6 TTTTTTgRNA scaffold

U6 target gRNA expression vector

Step 5: Clone the synthesized gBlock into an empty backbone vector such as pCR-Blunt II-TOPO from Invitrogen (http://products.invitrogen.com/ivgn/product/K280020), or directly pcr amplify this fragment (gRNA_F: TGTACAAAAAAGCAGGCTTTAAAG, gRNA_R: TAATGCCAACTTTGTACAAGAAAG) for transfection and gRNA expression.

Prashant Mali (Church Lab), Version: Jan. 14, 2013

STEP 3: Incorporate 19 bp of the selected target sequence as highlighted here: 5′-NNNNN NNNNN NNNNN NNNNN NGG-3′ into two 60mer oligonucleotides as indicated below (sequences are 5′ to 3′, and the regions marked in green and red are reverse complements of each other):

Insert_F:
TTTCTTGGCTTTATATATCTTGTGGAAAGGACGAAACACCG
NNNNNNNNNNNNNNNNNNN
Insert_R:
GACTAGCCTTATTTTAACTTGCTATTTCTAGCTCTAAAACN
NNNNNNNNNNNNNNNNNNC

Step 4: Anneal the two oligos and extend these to make a 100 bp double stranded DNA fragment using Phusion polymerase (http://www.neb.com/nebecomm/products/productm0530.asp).

Step 5: Linearize the gRNA cloning vector (http://www.addgene.org/41824/) using AflII and incorporate the 100 bp DNA fragment from Step 4 above into it using Gibson assembly (http://www.neb.com/nebecomm/products/productE2611.asp). The resulting vector is the desired gRNA expression vector. Note: This synthesis strategy is amenable to construction of multiple gRNA expression vectors, and even large gRNA libraries using oligonucleotide pools synthesized using custom DNA arrays

(http://www.sciencemag.org/content/suppl/2013/01/03/science.1232033.DC1/Mali.SM.pdf).

Option A Option B

### Cas9 mRNA Production and Purification

In vitro-transcribed (IVT) mRNA is another transient delivery system for nucleases. IVT mRNA minimizes the risk of genome insertion, and it bypasses the requirement of nuclear entry for transcription, resulting in efficient genome editing. The short but robust expression of mRNA results in high activity and potentially low off-target effects. In the case of genome editing, half-lives of both the nuclease mRNA and its protein products are critical determinants for editing efficiency. As a result, the short half-life of mRNA may be a limitation for its use in some cases. To overcome this drawback, various modifications have been introduced into the structural elements of mRNA. The incorporation of anti-reverse m⁷GpppG cap analog, poly(A) tail, modified nucleotides, 5′ and 3′ UTRs containing regulatory elements, and synonymously frequent codons into mRNA exhibited enhanced stability and translational_efficiency in IVT mRNA. As an alternative to linear mRNA, exogenous synthesized circular RNA (circRNA) has been developed for robust and stable protein expression. In addition, protein expression kinetics also have contributed to the editing efficiency of mRNA encoding nucleases. When delivered, protein expression of mRNA starts almost transiently, peaks within several hours (˜6-24 h), and persists for a few days. The expression duration is long enough for genome editing. Like DNA, the exposure of mRNA in the cytoplasm may induce an immune response, which remains a key barrier for mRNA-based genome editing. It has been shown that nucleotide modifications, sequential engineering, and high-performance liquid chromatography (HPLC)-purified mRNA resulted in lower immunogenicity and higher stability than unmodified nucleotides. Therefore, mRNA delivery of nucleases holds great potential for therapeutic application.

### In Vitro Transcription

### Plasmid DNA (pDNA) Preparation for mRNA Synthesis

For mRNA-based vaccine design, in vitro transcription of a plasmid DNA (pDNA) template is typically used to produce functional synthetic mRNA. The plasmid vector usually contains the following elements: an upstream promoter exclusively recognized by T7, SP6 or T3 RNA polymerase, all of which are derived from bacteriophages; 5′ UTR, cDNA, 3′ UTR, a downstream poly A-tail; and a unique cleavage site downstream of the poly A-tail (refer to FIG. 4 in this application).

FIG. 4. Plasmid generation for in vitro transcription (IVT). Following gene synthesis, cloning of the target DNA uses a plasmid vector (pDNA) for Cas9. The pDNA_CAS9 is expanded in bacterial culture, then purified using nucleic acid purification methods, such as silica-based membranes in spin columns.

### mRNA In Vitro Transcription and Capping

Bacteriophage RNA polymerase is normally used to transcribe linearized plasmid DNA. The pDNA is first linearized with the selected unique restriction site enzyme. After digestion, the linearized pDNA may be purified using methods such as the phenol-chloroform protocol, or the GenElute PCR Clean-Up Kit. For large scale purification, tangential flow filtration (TFF) is often advisable, as it can be easily scaled up. Following linearization, in vitro transcription and capping is performed in a mixed solution of recombinant RNA polymerase (T7, T3 or SP6) and nucleoside triphosphates, plus a cap analog such as CleanCap® Reagent or ARCA (Anti-Reverse Cap Analog). The modified nucleoside such as N1-Methylpseudouridine-5′-Triphosphate (N1-Methylpseudo-UTP, 1-Methylpseudo-UTP) can be used instead of GTP to suppress the innate immune system, and is used in the current mRNA vaccines. The efficiency of capping mRNA with ARCA is 70˜80% on average because it competes with GTP, while the yield of mRNA is reduced by about one-fourth compared to standard mRNA synthesis. In comparison, the CleanCap® reagent has been shown to work at 94% capping efficiency without affecting the yield of mRNA production. Alternatively, capping may be achieved by performing the transcription without a cap analog, instead employing the vaccinia virus-encoded capping complex (capping enzyme, 2′-O-Methyltransferase, GTP, and S-adenosyl methionine (SAM)). Capping efficiency will differ depending on the secondary structure of the mRNA of interest. Finally, when the length of the poly A tail of the template pDNA is insufficient (up to 150 bases), it can be extended by use of poly A enzyme (seer FIG. 5).

FIG. 5. mRNA synthesis is completed by linearization of pDNA, in vitro transcription (IVT) of mRNA using cell-free methods, and capping of the mRNA using cap analog or virus-encoded capping complex.

Cas9 mRNA. Cas9 mRNA (TriLink Biotechnologies) and sgRNA were coloaded into 306-O12B LNPs and intravenously injected into female Ai14 mice at a dose of 1.65 mg/kg total RNA.

### Lipid Nanoparticle Formulation

To formulate LNP for gene knock-out (LNP-RNP), sgRNA and Cas9_mRNA were mixed at a 1:1 molar ratio in different formulation buffers (100 mM citrate buffer (pH 4.0), Dulbecco's PBS (pH 7.4), 50 mM HEPES buffer (pH 7.4; LNP-RNP [HEPES]), or nuclease-free water at an RNP concentration of 0.4 μM. Complexation was performed for 15 minutes at room temperature. Concurrently, the lipids were mixed together in ethanol to achieve a total lipid to sgRNA ratio of 40:1 (w/w), resulting in a total lipid weight of 9.6 μg. The RNP and lipids were mixed by pipetting at a volume ratio of 3:1 (18 μl RNP to 6 μl lipids), and incubating for 15 minutes at room temperature. Subsequently, the formulation was diluted 4 times with PBS to a final RNP molar concentration of 76.9 nM in 100 μl.

### Physical Characterization of Lipid Nanoparticles

LNP were diluted 1.3 times further in 1×PBS for characterization of size and poly-dispersity index (PDI) using a Zetasizer Nano S (Malvern ALV CGS-3, the United Kingdom). The ζ-potential was determined with Zetasizer Nano Z (Malvern ALV CGS-3, United Kingdom) after 9× dilution in 10 mM HEPES buffer at pH 7.4. Each sample was measured in triplicate to determine size and ζ-potential two days after formulation.

Quantification of RNP complexed with LNP Complexation efficiencies were determined in LNP prepared in the different formulation conditions. RNP at 1.25 μM and a final formulation volume of 0.47 mL in PBS were used. For determination of Cas9 complexation, the LNP formulation was additionally dialyzed against 1×HBS with Float-A-Lyzer MWCO 300 kDa dialysis chambers (Avan-tor®, the Netherlands) to remove free SpCas9 from the formulation. The Quant-iT™ RiboGreen® RNA kit (Fisher Scientific, Landsmeer, the Netherlands) was used to determine the complexation efficiency of sgRNA. The protocol provided by the supplier was followed, except that sgRNA was used instead of the RNA standard to generate a calibration curve in RNAse-free TE buffer. A calibration curve with and without 2% Triton X-100 was made in duplicate. LNP samples and the calibration curve that were not treated with 2% Triton X-100 were treated with the same volume of 1×RNAse-free TE buffer. Fluorescence signal (ex. 485 nm, em. 520 nm) was determined with Jasco FP8300 Spectrofluorometer with micro-well plate reader (JASCO Benelux BV., De Meern, the Netherlands).

### Delivery of Specific LNP to Brain

There are several routes of administration that can access to brain. One example is intrathecal delivery and/or delivery to cerebrospinal fluid (CSF). There is also capability to inject very small volume of genome editing products directly into different regions of brain including Putnam, frontal lobe, cortex region. The volume injected is approximately below 100 microliter and infused slowly into different regions of brain. The most commonly used device can be purchased from clearpoint. This device allows for precision delivery of the genome editing materials mixed with contrasting agents in different location of brain using MRI (see FIG. 6).

### Sequencing Analysis to Detect SNP

For detection of SNPs several methods can be use that include direct sequencing of target SNPs following primer specific Quantitative Polymerase Chain Reaction referred here as qPCR.

### Description of Figures, Excel Documents and Tables

Column 1: Identifier for each SNP targeted
Column 2: The identifier for chromosome harboring the target SNP
Column 3: The exact location of the target gene
Column 4: The exact strand of DNA (−) versus (+)
Column 5: The identity of nucleotide (AGCT) which is expressed in healthy individuals referred to as reference sequence
Column 6: The sequence of polymorphism found in patients with severe mental health disease
Column 7: The sequence surrounding the nucleotide mutated in individuals with severe mental health disease
Column 8: gRNA sequence
Column 9-11: Measure of quality of representative gRNA specificity and off target probability
Column 12: Location of edit for example in exon, intron or intergenic regions.

The excel document II includes the following columns:
Column 1: SEQID of target genes SNP
Column 2: Sequence information providing at least 30 nucleotides flanking the 3 and 5′ or gRNA sequence forward or reverse

This is a reference to ASC II test file which was added in response to missing part that contains SEQID for all sequences provided in this patent application

The file name is SequenceID_Patent(1).text corresponds to The excel document II

The size is 3.96 KB, 4KB on disk

Date created Jun. 20, 2022 at 10:59 AM

The file name is Target_Gene_SNP_Corijan corresponds to the excel document I

The size is 4.96 KB, 9.00 KB on disk

Date created Jun. 20, 2022, 11:04 AM

Table I includes targets other than SNPs specified in this patient application.

LENGTHY TABLES
The patent application contains a lengthy table section. A copy of the table is available in electronic form from the USPTO web site (https://seqdata.uspto.gov/?pageRequest=docDetail&DocID=US20240011053A1). An electronic copy of the table will also be available from the USPTO upon request and payment of the fee set forth in 37 CFR 1.19(b)(3).

FIG. 1. Mechanism by which a complex between nuclease and guide RNA can be used to change DNA.

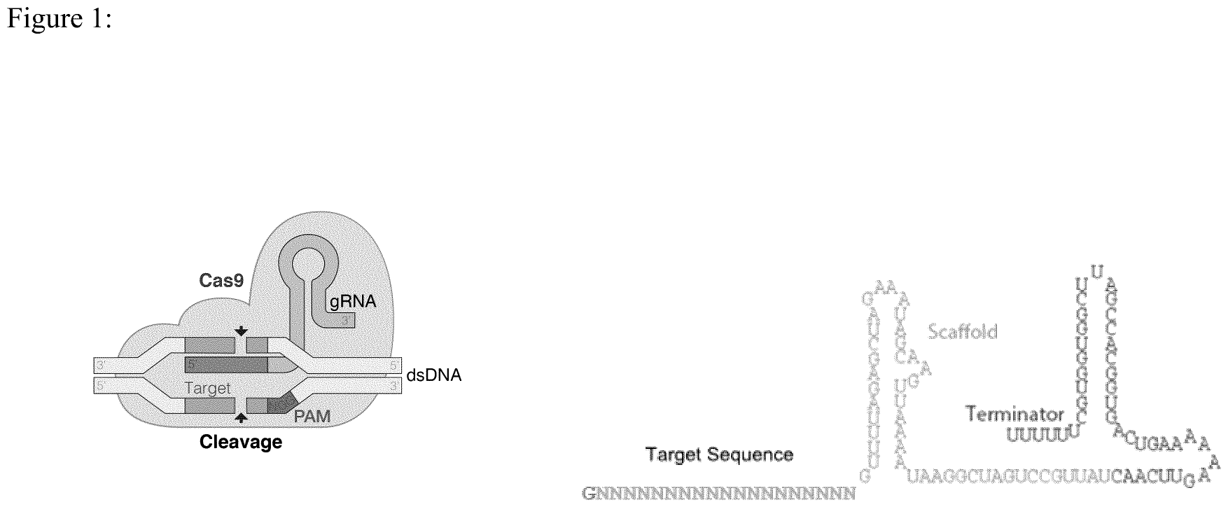

FIG. 2. Various methods for delivering CRISPR-Cas9 components for in vivo genome editing. Schematic illustrations represent varieties of the tools to deliver genome editing components. The delivery tools are roughly classified into three categories: biological, using viruses to deliver gene editing reagents. These viruses include AdV, AAV, Retroviruses or Lentiviruses and Virus like particles. The chemical method which is described here involves making a lipid nanoparticle which is loaded with components of gene editing reagents that include gRNA and Cas9 endonuclease protein or mRNA.

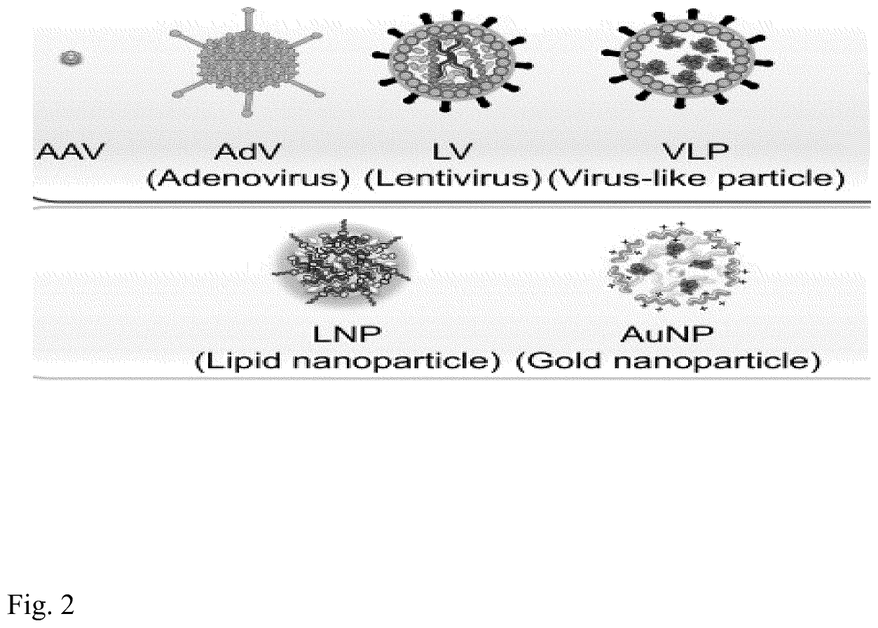

FIG. 3: Schematic showing LNP delivery into brain and uptake by neuronal cells and edit of target SNP

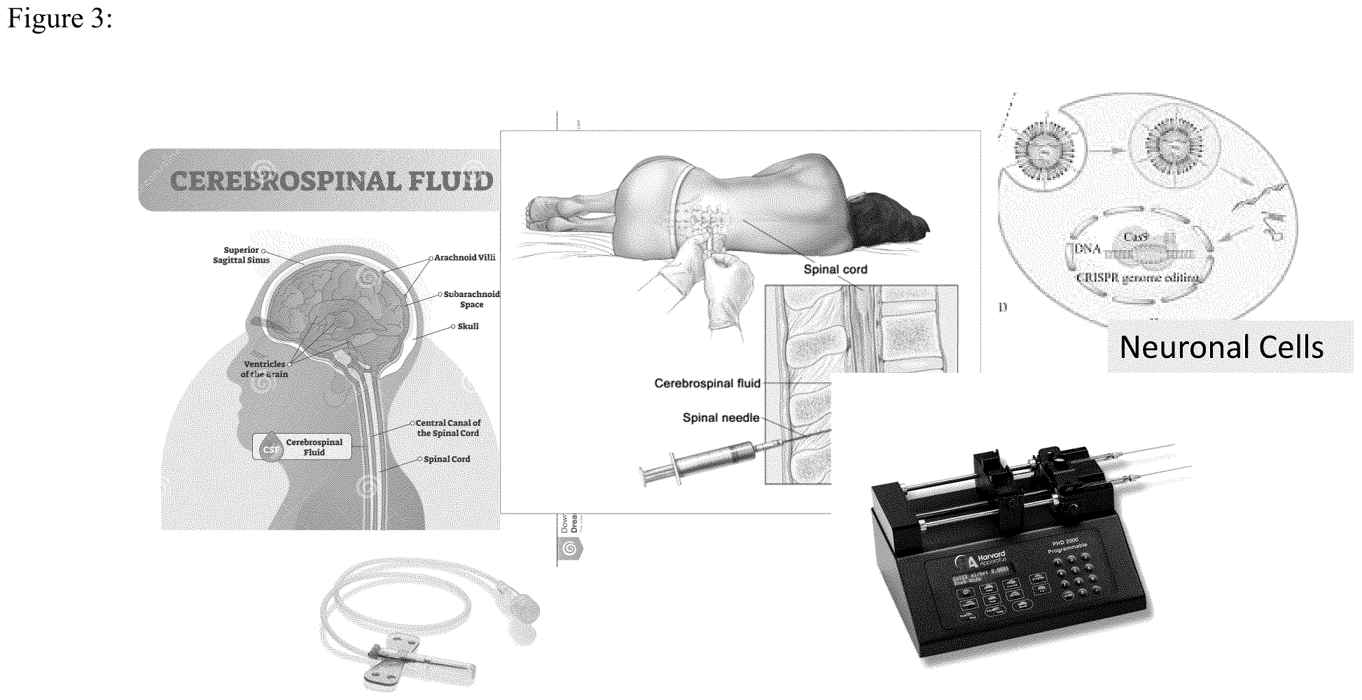

FIG. 4. Plasmid generation for in vitro transcription (IVT). Following gene synthesis, cloning of the target DNA uses a plasmid vector (pDNA) for Cas9. The pDNA_CAS9 is expanded in bacterial culture, then purified using nucleic acid purification methods, such as silica-based membranes in spin columns

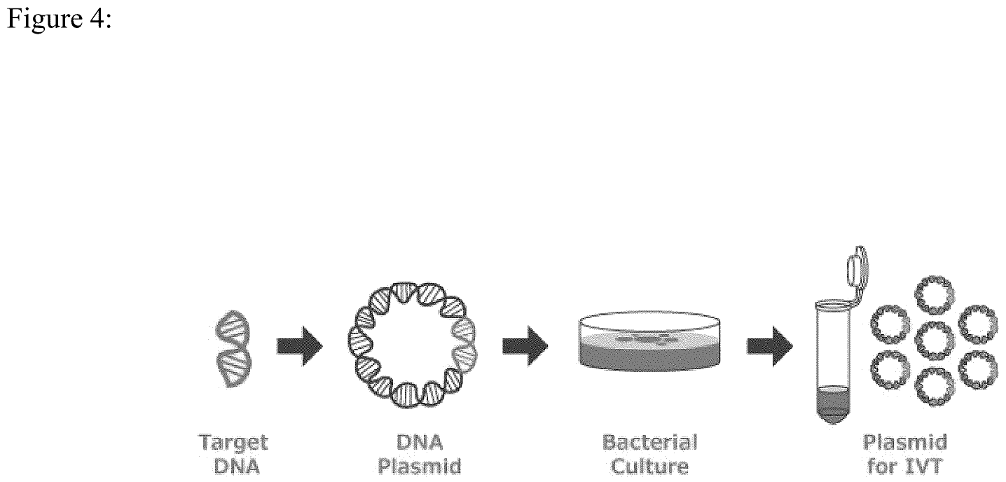

FIG. 5. mRNA synthesis is completed by linearization of pDNA, in vitro transcription (IVT) of mRNA using cell-free methods, and capping of the mRNA using cap analog or virus-encoded capping complex.

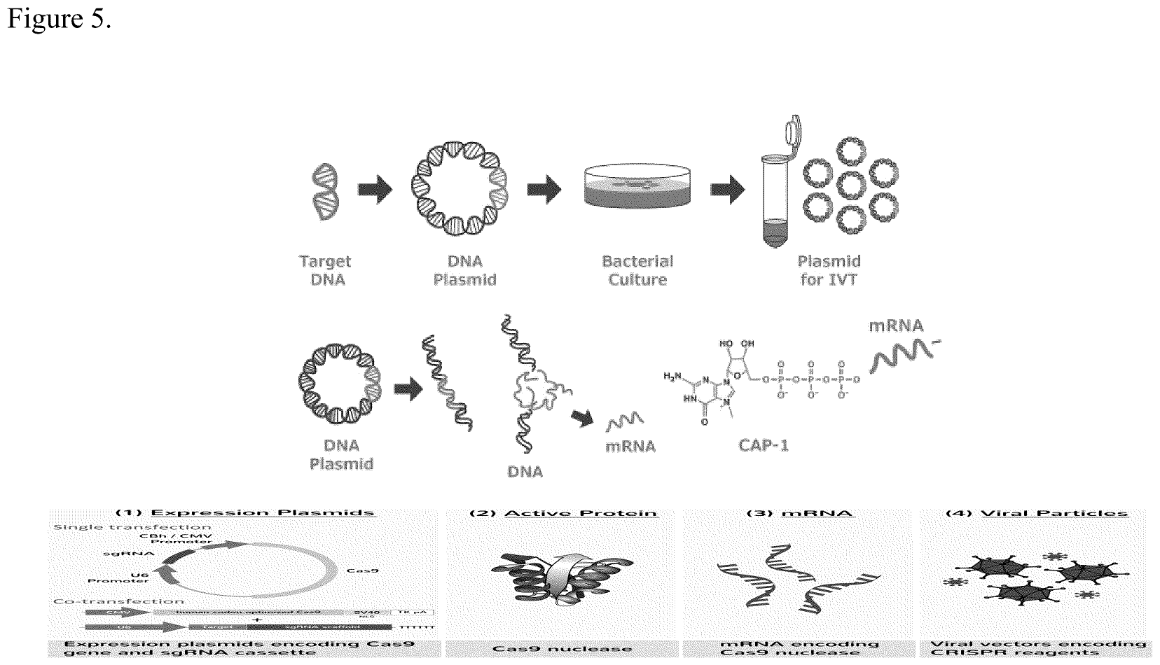

FIG. 6: Example of deliver device (Clearpoint) allowing region specific delivery of genome editing materials

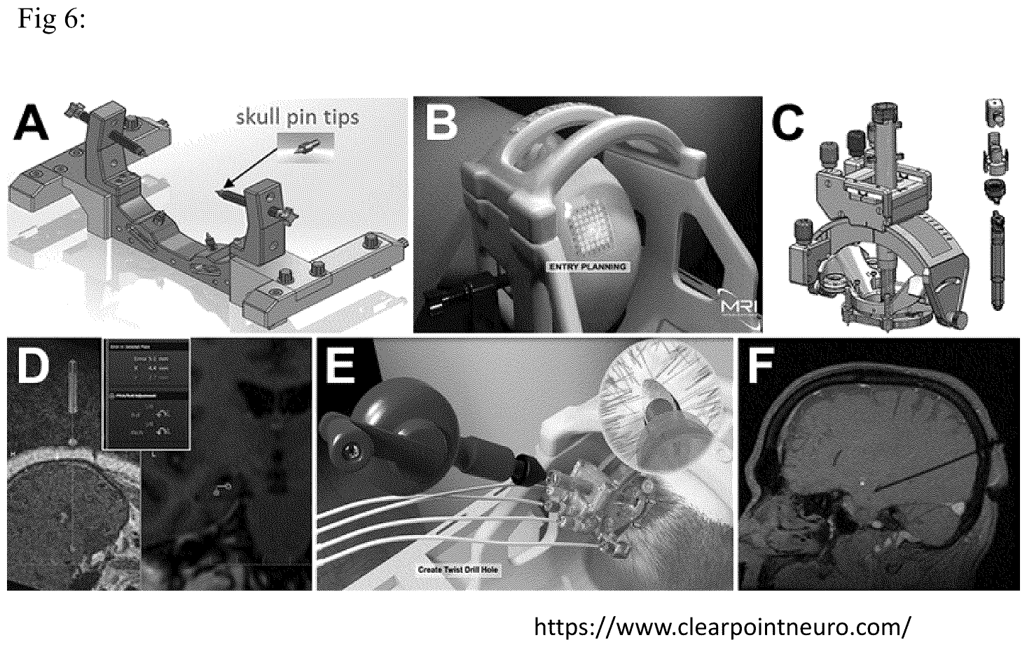

Cross Reference to Provisional Patent Application: 63/203,017 submitted Jul. 14, 2021 confirmation number 8078

The following patents are also referenced:
U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233, 8,999,641, 9,499,847, 9,938,521, 10,253,312, 11,028,388, 2007/0020627, 2010/0055793, 2010/0055798, 2010/0076057, 2011/0223638, 2012/0270273, 2013/0253040, 2014/0068797, 2014/0179770, 2014/0242699, 2014/0309177, 2014/0315985, 2014/0335620, 2014/0342456, 2014/0342457, 2014/0342458, 2014/0356958, 2015/0056705, 2015/0232833, 2015/0252358, 2015/0259704, 2016/0281111, 2016/03224987, 2016/0340661, 2017/0058298, 2018/0291370

### REFERENCES

### Mental Health Disease References

Juliann M. Savatt, Genetic Testing in Neurodevelopmental Disorders, Front. Pediatr., 19 Feb. 2021.

Nimah Mullins et al., Dissecting the Shared Genetic Architecture of Suicide Attempt, Psychiatric Disorders, and Known Risk Factors, Biological Psychiatry Volume 91, Issue 3, 1 Feb. 2022, Pages 313-327

H Le-Niculescu, Precision medicine for mood disorders: objective assessment, risk prediction, pharmacogenomics, and repurposed drugs, Mol Psychiatry, 2021 July; 26 (7):2776-2804

Zai C C, de Luca V, Strauss J, et al. Genetic Factors and Suicidal Behavior. In: Dwivedi Y, editor. The Neurobiological Basis of Suicide. Boca Raton (FL): CRC Press/Taylor & Francis; 2012. Chapter 11.

Brenda Cabrera-Mendoza, Brain Gene Expression Profiling of Individuals With Dual Diagnosis Who Died by Suicide, Comparative Study

J Dual Diagn, April-June 2020; 16 (2):177-190

Karlsson Linnér, R., Mallard, T. T., Barr, P. B. et al. Multivariate analysis of 1.5 million people identifies genetic associations with traits related to self-regulation and addiction. Nat Neurosci 24, 1367-1376 (2021).

### OTHER APPLICABLE REFERENCES

Zetsche, B., et al., “Multiplex Gene Editing by CRISPR-Cpf1 Through Autonomous Processing of a Single crRNA Array,” Nat. Biotechnol. 35 (1) 31-34 (2017).

Maeder, M. L., et al., “Development of a gene-editing approach to restore vision loss in Leber congenital amaurosis type 10,” Nat. Med. 25 (2):229-233 (2019).

Paix, A., et al., “Precision Genome Editing Using CRISPR-Cas9 and Linear Repair Templates in C. Elegans,” Methods 121-121:86-93 (2017).

Al-Attar, S., et al., “Clustered Regularly Interspaced Short Palindromic Repeats (CRISPRs): The Hallmark of an Ingenious Antiviral Defense Mechanism in Prokaryotes,” Biol. Chem. 392:277-289 (2011).

Altschul, S. F., et al., “Basic Local Alignment Search Tool,” J. Mol. Biol. 215 (3):403-410 (1990).

Amrani, N., et al., “NmeCas9 is an Intrinsically High-Fidelity Genome-Editing Platform,” Genome Biol. 19:214 (2018).

Anders, C., et al., “Structural Basis of PAM-Dependent Target DNA Recognition by the Cas9 Endonuclease,” Nature 513 (7519):569-573 (2014).

Bae, S., et al., “Cas-OFFinder: A Fast and Versatile Algorithm that Searches for Potential Off-Target Sites of Cas9 RNA-Guided Endonucleases,” Bioinformatics 30 (10):1473-1475 (2014).

Baker, M., “Gene Editing at CRISPR Speed,” Nat. Biotechnol. 32 (4):309-312 (2014).

Barrangou, R., “RNA-Mediated Programmable DNA Cleavage,” Nat. Biotechnol. 30 (9):836-838 (2012).

Barretina, J., et al., “The Cancer Cell Line Encyclopedia Enables Predictive Modeling of Anticancer Drug Sensitivity,” Nature 483 (7391):603-607 (2012).

Bassett, A. R., et al., “CRISPR/Cas9 and Genome Editing in Drosophila,” J. Genet. Genom. 41:7-19 (2014).

Bhaya, D., et al., “CRISPR-Cas Systems in Bacteria and Archaea: Versatile Small RNAs for Adaptive Defense and Regulation,” Annu. Rev. Genet. 45:273-297 (2011).

Bikard, D., et al., “Programmable Repression and Activation of Bacterial Gene Expression Using an Engineered CRISPR-Cas System,” Nucl. Acids Res. 41 (15):7429-7437 (2013).

Bothmer, A., et al., “Characterization of the Interplay Between DNA Repair and CRISPR/Cas9-Induced DNA Lesions at an Endogenous Locus,” Nat. Commun. 8:13905 (2017).

Briner, A. E., et al., “Guide RNA Functional Modules Direct Cas9 Activity and Orthogonality,” Mol. Cell 56 (2):333-339 (2014).

Brummelkamp, T. R., et al., “A System for Stable Expression of Short Interfering RNAs in Mammalian Cells,” Science 296 (5567):550-553 (2002).

Burstein, D., et al., “New CRISPR-Cas Systems from Uncultivated Microbes,” Nature 542 (7640):237-241 (2017).

Caldecott, K. W., “Single-Strand Break Repair and Genetic Disease,” Nat. Rev. Genet. 9 (8):619-631 (2008).

Canver, M. C., “Evaluation of the Clinical Success of Ex Vivo and In Vivo Gene Therapy,” Journal of Young Investigators, http://www.hyi.org/issue/evaluation-of-the-clinical-success-of-ex-vivo-an-d-in-vivo-gene-therapy/, 9 pages (2009).

Carroll, D., “A CRISPR Approach to Gene Targeting,” Mol. Ther. 20 (9):1658-1660 (2012).

Cassini, A., et al., “A Highly Specific SpCas9 Variant is Identified by In Vivo Screening in Yeast,” Nat. Biotechnol. 36 (3):265-271 (2018).

Cathomen, T., et al., “Zinc-Finger Nucleases: The Next Generation Emerges,” Mol. Ther. 16:1200-1207 (2008).

Cermak, T., et al., “Efficient Design and Assembly of Custom TALEN and Other TAL Effector-Based Constructs for DNA Targeting,” Nucl. Acids Res. 39 (12):e82 (2011).

Chen, F., et al., “Targeted Activation of Diverse CRISPR-Cas Systems for Mammalian Genome Editing Via Proximal CRISPR Targeting,” Nat. Commun. 8:14958 (2017).

Chen, J. S., et al., “Enhanced Proofreading Governs CRISPR-Cas9 Targeting Accuracy,” Nature 550 (7676):407-410 (2017).

Cho, S. W., et al., Supplementary Information: Targeted Genome Engineering in Human Cells With the Cas9 RNA-Guided Endonuclease, Nature Biotechnology (March 2013) vol. 31, No. 3, 11 pages.

Cho, S. W., et al., “Targeted Genome Engineering in Human Cells with the Cas9 RNA-Guided Endonuclease,” Nat. Biotechnol. 31 (3):230-232 (2013).

Christian, M., et al., “Targeting DNA Double-Strand Breaks With TAL Effector Nucleases,” Genetics 186:757-761 (2010).

Christian, M., et al., “Targeting DNA Double-Strand Breaks With TAL Effector Nucleases,” Genetics Supporting Information, 1SI-8SI (2010).

Cong, L., et al., “Multiplex Genome Engineering Using CRISPR/Cas Systems,” Science 339 (6121):819-823 (2013).

Cong, L. et al., “Supplementary Material: Multiplex Genome Engineering Using CRISPR-Cas Systems,” Science Express (Jul. 5, 2012).

Cong, L. et al., “Supplementary Material: Multiplex Genome Engineering Using CRISPR-Cas Systems,” Science Express (Jan. 3, 2013).

Esvelt, K. M., et al., “Orthogonal Cas9 Proteins for RNA-Guided Gene Regulation and Editing,” Nat. Methods 10 (11):1116-1121 (2013).

Grieger, J. C., et al., “Production and Characterization of Adeno-Associated Viral Vectors,” Nat. Protoc. 1 (3):1412-1428 (2006).

Hatoum-Aslan, A., et al. “Mature Clustered Regularly Interspaced, Short Palindromic Repeats RNA 5,9,14 (crRNA) Length is Measured by a Ruler Mechanism Anchored at the Precursor Processing Site,” Proc. Natl. Acad. Sci. 108 (52):21218-21222 (2011).

Heigwer, F., et al., “E-CRISP: Fast CRISPR Target Site Identification,” Nat. Methods 11 (2):122-123 (2014).

Hockemeyer, D., et al., “Efficient Targeting of Expressed and Silent Genes in Human ESCs and iPSCs Using Zinc-Finger Nucleases,” Nat. Biotechnol. 27 (9):851-857 (2009).

Hockemeyer, D., et al., “Genetic Engineering of Human Pluripotent Cells Using TALE Nucleases,” Nat. Biotechnol. 29:731-734 (2011).

Hsu, P. D., et al., “DNA Targeting Specificity of RNA-Guided Cas9 Nucleases,” Nat. Biotechnol. 31 (9):827-832 (2013).

Jiang, W., et al., “RNA-Guided Editing of Bacterial Genomes Using CRISPR-Cas Systems,” Nat. Biotechnol. 31 (3):233-239 (2013).

Jinek, M., et al., “A Programmable Dual-RNA-Guided DNA Endonuclease in Adaptive Bacterial Immunity,” Science 337 (6096):816-821 (2012).

Jinek, M., et al., “Structures of Cas9 Endonucleases Reveal RNA-Mediated Conformational Activation,” Science 343 (6176):1247997 (2014).

Jinek, M., et al., “RNA-Programmed Genome Editing in Human Cells,” eLife 2:e00471 (2013).

Kleinstiver, B. P., et al., “Broadening the Targeting Range of Staphylococcus aureus CRISPR-Cas9 by Modifying PAM Recognition,” Nat. Biotechnol. 33 (12):1293-1298 (2015).

Kleinstiver, B. P., et al., “Engineered CRISPR-Cas9 Nucleases with Altered PAM Specificities,” Nature 523 (7561):481-485 (2015).

Kleinstiver, B. P., et al., “High-Fidelity CRISPR-Cas9 Nucleases with No Detectable Genome-Wide Off-Target Effects,” Nature 529 (7587):490-495 (2016).

Lee, J. K., et al., “Directed evolution of CRISPR-Cas9 to Increase Its Specificity,” Nat. Commun. 9:3048 (2018).

Tool for Engineering Biology,” Nat. Methods 10 (10):957-963 (2013). cited by applicant . . .

Nishimasu, H., et al., “Crystal Structure of Cas9 in Complex with Guide RNA and Target DNA,” Cell 156 (5):935-949 (2014).

Peng, R., et al., “Potential Pitfalls of CRISPR/Cas9-Mediated Genome Editing,” FEBS J. 283:1218-1231 (2016).

Purnick, P. E. M., et al., “The Second Wave of Synthetic Biology: From Modules to Systems,” Nat. Rev. Mol. Cell Biol. 10 (6):410-422 (2009).

Shen, B., et al., “Generation of Gene-Modified Mice via Cas9/RNA-Mediated Gene Targeting,” Cell Res. 23:720-723 (2013).

Sternberg, S. H., et al., “DNA Interrogation by the CRISPR RNA-Guided Endonuclease Cas9,” Nature 507 (7490):62-67 (2014).

Strecker, J., et al., “Engineering of CRISPR-Cas12b for Human Genome Editing,” Nat. Commun. 10:212 (2019).

Tsai, S. Q., et al., “Open-Source GuideSeq Software for Analysis of GUIDE-Seq Data,” Nat. Biotechnol. 34 (5):483 (2016).

Wang, J., et al., “Highly Efficient Homology-Driven Genome Editing in Human T Cells by Combining Zinc-Finger Nuclease mRNA and AAV6 Donor Delivery,” Nucleic Acids Res. 44 (3):e30 (2016).

Wang, T., et al., “Genetic Screens in Human Cells Using the CRISPR-Cas9 System,” Science 343 (6166):80-84 (2013).

Wang, J., et al., “xCas9 Expands the Scope of Genome Editing with Reduced Efficiency in Rice,” Plant Biotechnol. J. 17:709-711 (2019).

Wu, X., et al., “Genome-Wide Binding of the CRISPR Endonuclease Cas9 in Mammalian Cells,” Nat. Biotechnol. 32 (7):670-676 (2014).

Wu, Y., et al., “Correction of a Genetic Disease in Mouse via Use of CRISPR-Cas9,” Cell Stem Cell 13 (6):659-662 (2013).

Xiao, A., et al., “CasOT: A Genome-Wide Cas9/gRNA Off-Target Searching Tool,” Bioinformatics 30 (8):1180-1182 (2014).

Mukherjee-Clavin, B., et al., “Current Approaches for Efficient Genetic Editing in Human Pluripotent Stem Cells,” Front. Biol. 8 (5):461-467 (2013).

Giannoukos, G., et al., “UDiTaS™, a genome editing detection method for indels and genome rearrangements,” BMC Genomics 19:212 (2018).

Kleinstiver, B. P., et al., “Engineered CRISPR-Cas12a variants with increased activities and improved targeting ranges for gene, epigenetic and base editing,” Nat. Biotechnol. 37 (3):276-282 (2019).

Strohkendl, I., et al., “Kinetic Basis for DNA Target Specificity of CRISPR-Cas12a,” Mol Cell. 71 (5):816-824 (2018).

1. Method of treating and curing severe mental disease attributed by SNPs or genetic biomarkers using genome editing technologies

2. Method of treatment by targeting 1 or more SNPs outlined in Excel document I of this application and herein and any other SNPs discovered to be positively correlated with severe mental health diseases. SNPs
Identifier, rs4851921, rs9310709, rs53576, rs6449197, rs6295, rs71557378, rs62474683, rs745527286, rs10994330, rs1938526, rs6265, rs2298526, rs242939, rs242941, rs4309482, rs12966547 rs4680

3. Method of treatment of severe mental health disease by targeting a single SNPs outlined in claim 2.

4. Method of treatment of mental health disease by targeting any of the following sequences containing SNP which is identified in capital letter that is associated with severe mental health disease. Sequence of target DNA, SNP position is identified in capital letter
gatattccatgtggtgtggggctggagaagcctgggcgcc
actggtggagGcagcccgaggcaggcacaacataaccgag
gacagcaagactccctcagaa,
aatgaatgtctctaccccaaactatgagagctccaggggc
agagacttgttattttgtccatTcttatccccagagtttg
gcaactggtcagcacttgataaataaaggaaagaatgt,
ctcgggcacagcattcatggaaaggaaaggtgtacgggac
atgcccgaggAtcctcaagtcccacagaaacagggagggg
ctggggaagctcattctacagatg,
ctcgggcacagcattcatggaaaggaaaggtgtacgggac
atgcccgaggAtcctcaagtcccacagaaacagggagggg
ctggggaagctcattctacagatg,
tggggtttaatagatgaattttatcaggttgaggaaattt
tatttctaatCtgctcagtgttttttcatcacaagagtgt
tggattttgttaatatttttgt,
tcagtctcccaattattgctaattgatggaagaagaagac
cgagtgtggtcttcCtttttaaaaagctacctccgttctc
gcgccattgcactccagctgggcgac,
tcagtctcccaattattgctaattgatggaagaagaagac
cgagtgtggtcttcCtttttaaaaagctacctccgttctc
gcgccattgcactccagctgggcgac,
cctttgcttatttagcttctaatccattaatcatggagag
cttcattttttGGgggggggggggggggggggacttctca
gacctcatcaaactctactctaaagcactacca,
gcgcaccgccctctcctccccteggccgcagtccccgcge
gccccgaggegTgcttgccctccgccaagegegcccacta
ccctgcccgctcctgcagggggcta,
ctcgtgggaacctcttattctgtttatgactcctcagcgg
tgcagaaagTtattccttcccttgctggacaccacatcaa
aggaggcccacaggtgtttcttccttc,
ccagagagcaaggccctggccaggaaggtgtcctgcaagc
tgtcgctgcgCcagcccggggaggtgagtgtgtgggctgg
gcagtccttatggtcatgctaagctggaggc,
agaaagcatggtacagtggggaaggtacatggatggatga
agatggccatgacaAtgagattaagagacaccatggtgag
agagcttcacaaagcc,
ttcttcattgggccgaactttctggtcctcatccaacagc
tcttctatcaCgtgtcgaaagtgtcagccaatgatgtcaa
gcctcttga,
acttagagtaggcatggcagagggagctaacccataggaa
attaaggaattTgaagaatctttgaaagttcatctcattt
aacttctttaaaacaaaatttgtgaa,
acttagagtaggcatggcagagggagctaacccataggaa
attaaggaattTgaagaatctttgaaagttcatctcattt
aacttctttaaaacaaaatttgtgaa,
ccgcccagcctcaggtttcagatctgagttggtcactcct
tcacttggaaCccactcttgtgtggcctccgtgttcaggc
tgctgggtggggccggccaggctgt,
ccgcccagcctcaggtttcagatctgagttggtcactcct
tcacttggaaCccactcttgtgtggcctccgtgttcaggc
tgctgggtggggccggccaggctgt,
gcttggcagctgctaaggccteggcccaggcctgaagagg
ctgcccccacAccgctggttcatggttcctggccctccgt
ggcttgtctctgtccactcttgtgcc,
gcttggcagctgctaaggccteggcccaggcctgaagagg
ctgcccccacAccgctggttcatggttcctggccctccgt
ggcttgtctctgtccactcttgtgcc,
acaacgatgtgtataaatctccaaaggcatcatgctaagt
gacaggagtcAgtctcaaaaagttacataccgtgtgattc
cattgatatgacatcctctaaaaga,
acaacgatgtgtataaatctccaaaggcatcatgctaagt
gacaggagtcAgtctcaaaaagttacataccgtgtgattc
cattgatatgacatcctctaaaaga,
cagttaagtgggagaaaaaataaaagtaaataacatttaa
taggacaagGatagtaagtcagagtgtggacttttcattg
actcacatgaggtctctgtttcaact,
cagttaagtgggagaaaaaataaaagtaaataacatttaa
taggacaagGatagtaagtcagagtgtggacttttcattg
actcacatgaggtctctgtttcaact,
tcaaccccgactgtgccgccatcacccagoggatggtggt
ggatttcgctggcGtgaaggacaaggtgtgcatgcctgac
ccgttgtcagacctggaaaaagggccgg,

5. Method of treatment of mental health disease by targeting one of the following sequences containing SNP identified in claim 4.

6. Method of treating severe mental health disease according to claim 2 using genome editing tools which are not limited to Cluster Interspersed Short Palindromes Repeat (CRISPR/Cas9), Zinc Finger Nucleases, Engineered meganucleases, Transcription Activator Like Effector Nucleases (TALENs), and other technologies such as base editors

7. Preferred method of claim 2 is by using CRISPR/Cas9, the bacterial endonuclease CAS9 is delivered into cells or into patient directly in complex with what is known as guide RNA. The gRNA is designed to have homology with region of target gene such that nuclease can initiate a repair in the target gene to correct the SNPs.

8. Preferred genome editing materials as described in claim 4 can be delivered in forms of mRNA for CAS9 and synthetic gRNA or in form of purified nuclease CAS9 in complex. with gRNA. The complex is delivered in complex with Lipid nanoparticles (LNP).

9. The complex in claim 8 can be administered to patients using several routes of deliveries. These include intrathecal delivery, delivery to cerebrospinal fluid directly or delivery into different regions of brain.

10. Alternatively, in another embodiment of claim 6 the virus AAV or lentivirus that encode for the functional protein can be used to deliver the normal gene and protein to the patient.

11. Preferred method of delivery of LNP/gRNA/CAS9 or AAV vector engineered to express the functional gene must be delivered to anatomy which is accessible to brain. It is understood that large particulate like gene editing complexes (CAS9/gRNA/LNP) and engineered AAV and lentiviruses can not efficiently cross blood brain barrier.

12. Treatment of severe mental health disease using genome editing and composition of 1 or more than 2 gRNAs identified in excel document #1 and herein in complex with CAS9 mRNA or protein delivered using lipid nanoparticles. gRNA Sequence,
AAATAACATTTAATAGGACAAGG,
GCTGACCAGTTGCCAAACTCTGGGGATAAGAA (rev),
AGGTGTACGGGACATGCCCGAGGATC,
AGGTGTACGGGACATGCCCGAGGATC
TTTAAAAAGGAAGACACACTCGG (rev),
TTTAAAAAGGAAGACACACTCGG (rev),
TCATGGAGAGCTTCATTTTTTGG,
GGAGAGCTTCATTTTTTGGGGGG,
CATGGAGAGCTTCATTTTTTGGG,
GGCGGAGGGCAGCACGCTCGGGG (rev),
TGGCGGAGGGCAGCACGCTCGGG
(rev),
ATGTGGTGTCCAGCAAGGGAAGGAATAA (rev),
AAGCTGTCGCTGCGCCAGCCCGG,
GCTGTCGCTGCGCCAGCCCGGGG,
AGCTGTCGCTGCGCCAGCCCGGG,
GTCTCTTAATCTCATTGTCATGG (rev),
ACACGTGATAGAAGAGCTGTTGG (rev),
AGCTAACCCATAGGAAATTAAGGAATTT,
AGCTAACCCATAGGAAATTAAGGAATTT,
ACTTGGAACCCACTCTTGTGTGG,
CACGGAGGCCACACAAGAGTGGG (rev),
ACTTGGAACCCACTCTTGTGTGG,
CACGGAGGCCACACAAGAGTGGG (rev),
GAACCATGAACCAGCGCGTGTGG,
ACCATGAACCAGCGCGTGTGGGG,
CCATGAACCAGCGCGTGTGGGGG,
AACCATGAACCAGCGCGTGTGGG
GAACCATGAACCAGCGCGTGTGG,
ACCATGAACCAGCGCGTGTGGGG,
CCATGAACCAGCGCGTGTGGGGG,
AACCATGAACCAGCGCGTGTGGG,
AAGGCATCATGCTAAGTGACAGGAGTCA,
AAGGCATCATGCTAAGTGACAGGAGTCA,
AAATAACATTTAATAGGACAAGG,
AAATAACATTTAATAGGACAAGG,
TTTCGCTGGCATGAAGGACAAGG

13. Method of treating mental health disease by targeting and converting sequence back to normal sequence referred to as reference sequence using 1 or more than 1 gRNAs described in claim 12 in complex with CAS9 mRNA or protein and delivered using lipid nanoparticles.

14. Method of treating mental health disease by targeting and converting sequence back to normal sequence referred to as reference sequence using a single gRNAs specified in excel document #1 and described in claim 12 in complex with CAS9 mRNA or protein and delivered using lipid nanoparticles

15. Method of treating mental health disease according to claim 14 by targeting SEQID1:gatattccatgtggtgtgggggctggagaagcctgggcgccactggtgagGcagcccgaggcaggcacacata accgaggacagcaagactccctcagaa where the sequence of SNP is in capital letter

16. Method of treating mental health disease according to claim 14 by targeting specific gene with target gene containing SNP rs4851921 on chromosome 2 at intergenic location of target gene:c2arf40, UXS1

17. Method of treating mental health disease according to claim 14 using gRNA with the composition of gRNA with SEQID2:AAATAACATTTAATAGGACAAGG

18. Method of treating mental health disease according to claim 14 by targeting SEQID3:aatgaatgtctctaccccaaactatgagagctccaggggcagagacttgttattttgtccatTcttatccccagagttt ggcaactggtcagcacttgataaataaaggaaagaatgt where the sequence of SNP is in capital letter

19. Method of treating mental health disease according to claim 14 by targeting specific gene with target SNP rs9310709 on chromosome 3 at intergenic location of target gene

20. Method of treating mental health disease using gRNA with the composition of gRNA with sequence SEQID4: GCTGACCAGTTGCCAAACTCTGGGGATAAGAA (rev)

21. Method of treating mental health disease according to claim 14 by targeting with SEQIDS:
ctcgggcacagcattcatggaaaggaaaggtgtacgggac
atgcccgaggAtcctcaagtcccacagaaacagggagggg
ctggggaagctcattctacagatg

22. Method of treating mental health disease according to claim 14 by targeting specific gene with the target SNP rs53576 at intron position of OXTR gene

23. Method of treating mental health disease according to claim 14 using gRNA with the composition of gRNA with SEQID6: AGGTGTACGGGACATGCCCGAGGATC

24. Method of treating mental health disease according to claim 14 by targeting with SEQID7:
ctcgggcacagcattcatggaaaggaaaggtgtacgggac
atgcccgaggAtcctcaagtcccacagaaacagggagggg
ctggggaagctcattctacagatg

25. Method of treating mental health disease according to claim 14 by targeting specific gene with the target SNP rs53576 on chromosome 3 in OXTR intron region

26. Method of treating mental health disease according to claim 14 using gRNA with the composition of gRNA with SEQID8: AGGTGTACGGGACATGCCCGAGGATC

27. Method of treating mental health disease targeting with SEQID9:
tggggtttaatagatgaattttatcaggttgaggaaattt
tatttctaatCtgctcagtgttttttcatcacaagagtgt
tggattttgttaatatttttgt

28. Method of treating mental health disease according to claim 14 by targeting specific gene with the target SNP rs6449197 on chromosome 4 in CD38 gene

29. Method of treating mental health disease according to claim 14 using gRNA with the composition of gRNA with SEQID10:TBD

30. Method of treating mental health disease according to claim 14 by targeting with SEQID11:
tcagtctcccaattattgctaattgatggaagaagaagac
cgagtgtggtcttcCtttttaaaaagctacctccgttctc
gcgccattgcactccagctgggcgac

31. Method of treating mental health disease according to claim 14 by targeting specific gene with the target SNP rs6295 on Chromosome 5 in HTR1A gene

32. Method of treating mental health disease according to claim 14 using gRNA with the composition of gRNA with SEQID12: TTTAAAAAGGAAGACACACTCGG (rev)

33. Method of treating mental health disease according to claim 14 by targeting with SEQID 13:
tcagtctcccaattattgctaattgatggaagaagaagac
cgagtgtggtcttcCtttttaaaaagctacctccgttctc
gcgccattgcactccagctgggcgac

34. Method of treating mental health disease according to claim 14 by targeting specific gene with the target SNP rs6295 on chromosome 5 of HTR1A gene

35. Method of treating mental health disease according to claim 14 using gRNA sequence SEQID14: TTTAAAAAGGAAGACACACTCGG (rev)

36. Method of treating mental health disease according to claim 14 targeting with SEQID15:
cctttgcttatttagcttctaatccattaatcatggagag
cttcattttttGGgggggggggggggggggggacttctca
gacctcatcaaactctactctaaagcactacc

37. Method of treating mental health disease according to claim 14 by targeting specific gene with the target SNP rs71557378 on chromosome 6 in the following region HIST1H2AA, HIS1H2BA

38. Method of treating mental health disease according to claim 14 using gRNA with the composition of gRNA with SEQID16: TCATGGAGAGCTTCATTTTTTGG or SEQID17: GGAGAGCTTCATTTTTTGGGGGG or SEQID18: CATGGAGAGCTTCATTTTTTGGG

39. Method of treating mental health disease according to claim 14 by targeting with SEQID19:
gcgcaccgccctctcctcccctcggccgcagtccccgcgc
gccccgaggcgTgcttgccctccgccaagcgcgcccacta
ccctgcccgctcctgcagggggcta

40. Method of treating mental health disease t according to claim 14 by targeting specific gene with the target SNP rs62474683 on chromosome 7

41. Method of treating mental health disease according to claim 14 using gRNA with the composition of gRNA with SEQID20: GGCGGAGGGCAGCACGCTCGGGG (rev) or SEQID21: TGGCGGAGGGCAGCACGCTCGGG (rev)

42. Method of treating mental health disease according to claim 14 by targeting SEQID22:
ctcgtgggaacctcttattctgtttatgactcctcagcgg
tgcagaaagTtattccttcccttgctggacaccacatcaa
aggaggcccacaggtgtttcttccttc

43. Method of treating mental health disease according to claim 14 by targeting specific gene with the target SNP rs745527286 on chromosome 9 in CFAP77 gene

44. Method of treating mental health disease according to claim 14 using gRNA with the composition if gRNA with SEQID23: ATGTGGTGTCCAGCAAGGGAAGGAATAA (rev)

45. Method of treating mental health disease according to claim 14 by targeting SEQID24:
ccagagagcaaggccctggccaggaaggtgtcctgcaagc
tgtcgctgcgCcagcccggggaggtgagtgtgtgggctgg
gcagtccttatggtcatgctaagctggaggc

46. Method of treating mental health disease according to claim 14 by targeting specific gene with the target SNP rs10994330 on chromosome 10 in ANK3 gene

47. Method of treating mental health disease according to claim 14 using gRNA with the composition of gRNA with SEQID25: AAGCTGTCGCTGCGCCAGCCCG or SEQID26: GCTGTCGCTGCGCCAGCCCGGGG or SEQID27: AGCTGTCGCTGCGCCAGCCCGGG

48. Method of treating mental health disease according to claim 14 by targeting SEQID28:
agaaagcatggtacagtggggaaggtacatggatggatga
agatggccatgacaAtgagattaagagacaccatggtgag
agagcttcacaaagcc

49. Method of treating mental health disease according to claim 14 by targeting specific gene with the target SNP rs1938526 of ANK3 gene

50. Method of treating mental health disease according to claim 14 using gRNA with the composition of gRNA with SEQID29: GTCTCTTAATCTCATTGTCATGG (rev)

51. Method of treating mental health disease according to claim 14 by targeting SEQID30:
ttcttcattgggccgaactttctggtcctcatccaacagc
tcttctatcaCgtgtcgaaagtgtcagccaatgatgtcaa
gcctcutga

52. Method of treating mental health disease according to claim 14 by targeting specific gene with the target SNP rs6265 of BDNF on chromosome 11

53. Method of treating mental health disease according to claim 14 using gRNA with the composition of gRNA SEQID31: ACACGTGATAGAAGAGCTGTTGG (rev)

54. Method of treating mental health disease according to claim 14 by targeting SEQID32:
acttagagtaggcatggcagagggagctaacccataggaa
attaaggaattTgaagaatctttgaaagttcatctcattt
aacttctttaaaacaaaatttgtgaa

55. Method of treating mental health disease according to claim 14 by targeting specific gene with the target SNP rs2298526 of NCAM1 on chromosome 11

56. Method of treating mental health disease according to claim 14 using gRNA with the composition of gRNA with SEQID33: AGCTAACCCATAGGAAATTAAGGAATTT

57. Method of treating mental health disease according to claim 14 by targeting SEQID34:acttagagtaggcatggcagagggagctaacccataggaaattaaggaattTgaagaatctttgaaagttcatct catttaacttctttaaaacaaaatttgtgaa

58. Method of treating mental health disease according to claim 14 by targeting specific gene with the target SNP rs2298526 of NCAM1 on chromosome 11

59. Method of treating mental health disease according to claim 14 using gRNA with the composition of gRNA with SEQID35: AGCTAACCCATAGGAAATTAAGGAATTT

60. Method of treating mental health disease according to claim 14 by targeting SEQID36:
ccgcccagcctcaggtttcagatctgagttggtcactcct
tcacttggaaCccactcttgtgtggcctccgtgttcaggc
tgctgggtggggccggccaggctgt

61. Method of treating mental health disease according to claim 14 by targeting specific gene with the target SNP rs242939 on chromosome 17 in CRHR1

62. Method of treating mental health disease according to claim 14 using gRNA with the composition of gRNA with SEQID37: ACTTGGAACCCACTCTTGTGTGG or SEQID38: CACGGAGGCCACACAAGAGTGGG (rev)

63. Method of treating mental health disease according to claim 14 by targeting SEQID39:
ccgcccagcctcaggtttcagatctgagttggtcactcct
tcacttggaaCccactcttgtgtggcctccgtgttcaggc
tgctgggtggggccggccaggctgt

64. Method of treating mental health disease according to claim 14 by targeting specific gene with the target SNP rs242939 on chromosome 17 in CRHR1

65. Method of treating mental health disease according to claim 14 using gRNA with the composition of gRNA with SEQID40: ACTTGGAACCCACTCTTGTGTGG or SEQID41: CACGGAGGCCACACAAGAGTGGG (rev)

66. Method of treating mental health disease according to claim 14 by targeting SEQID42:
gcttggcagctgctaaggcctcggcccaggcctgaagagg
ctgcccccacAccgctggttcatggttcctggccctccgt
ggcttgtctctgtccactcttgtgcc

67. Method of treating mental health disease according to claim 14 by targeting specific gene with the target SNP rs242941 on chromosome 17 of CRHR1

68. Method of treating mental health disease according to claim 14 using gRNA with the composition of gRNA with SEQID43: GAACCATGAACCAGCGCGTGTGG or SEQID44: ACCATGAACCAGCGCGTGTGGGG or SEQID45 CCATGAACCAGCGCGTGTGGGGG or SEQID46: AACCATGAACCAGCGCGTGTGGG

69. Method of treating mental health disease according to claim 14 by targeting SEQID47:
gcttggcagctgctaaggcctcggcccaggcctgaagagg
ctgcccccacAccgctggttcatggttcctggccctccgt
ggcttgtctctgtccactcttgtgcc

70. Method of treating mental health disease according to claim 14 by targeting specific gene with the target SNP rs242941 on Chromosome 17 CRHR1

71. Method of treating mental health disease according to claim 14 using gRNA with the composition of gRNA with SEQID48: GAACCATGAACCAGCGCGTGTGG or SEQID49:ACCATGAACCAGCGCGTGTGGGG or SEQID50:CCATGAACCAGCGCGTGTGGGGG or SEQID51:AACCATGAACCAGCGCGTGTGGG

72. Method of treating mental health disease according to claim 14 by targeting SEQID52:
acaacgatgtgtataaatctccaaaggcatcatgctaagt
gacaggagtcAgtctcaaaaagttacataccgtgtgattc
cattgatatgacatcctctaaaaga

73. Method of treating mental health disease according to claim 14 by targeting specific gene with the target SNP rs4309482 on chromosome 18 of TCF4

74. Method of treating mental health disease according to claim 14 using gRNA with the composition of gRNA with SEQID53: AAGGCATCATGCTAAGTGACAGGAGTCA

75. Method of treating mental health disease according to claim 14 by targeting SEQID54:
acaacgatgtgtataaatctccaaaggcatcatgctaagt
gacaggagtcAgtctcaaaaagttacataccgtgtgattc
cattgatatgacatcctctaaaaga

76. Method of treating mental health disease according to claim 14 by targeting specific gene with the target SNP rs4309482 on chromosome 18 of TCF4

77. Method of treating mental health disease according to claim 14 using gRNA with the composition of gRNA with SEQID55: AAGGCATCATGCTAAGTGACAGGAGTCA

78. Method of treating mental health disease according to claim 14 by targeting SEQID56:
cagttaagtgggagaaaaaataaaagtaaataacatttaa
taggacaagGatagtaagtcagagtgtggacttttcattg
actcacatgaggtctctgtttcaact

79. Method of treating mental health disease according to claim 14 by targeting specific gene with the target SNP rs12966547 on Chromosome 18 TCF4

80. Method of treating mental health disease according to claim 14 using gRNA with the composition of gRNA with SEQID56:AAATAACATTTAATAGGACAAGG

81. Method of treating mental health disease according to claim 14 by targeting SEQID58:
cagttaagtgggagaaaaaataaaagtaaataacatttaa
taggacaagGatagtaagtcagagtgtggacttttcattg
actcacatgaggtctctgtttcaact

82. Method of treating mental health disease according to claim 14 by targeting specific gene with the target SNP rs12966547 on chromosome 18 TCF4

83. Method of treating mental health disease according to claim 14 using gRNA with the composition of gRNA SEQID59: AAATAACATTTAATAGGACAAGG

84. Method of treating mental health disease according to claim 14 by targeting SEQID60:
tcaaccccgactgtgccgccatcacccagcggatggggtg
gatttcgctggcGtgaaggacaaggtgtgcatgcctgacc
cgttgtcagacctggaaaaagggccgg

85. Method of treating mental health disease t according to claim 14 by targeting specific gene with the target SNP rs4680 on chromosome 22 of COMT

86. Method of treating mental health disease according to claim 14 using gRNA with the composition of gRNA with SEQID61:TTTCGCTGGCATGAAGGACAAGG

87. In another independent claim the promoter for gene encoding growth factors, cytokines neuropeptides and hormones Brain Derived Neurotrophic Factor (BDNF) Glial Derived Neurotrophic Factor (GDNF) Neurotrophic Growth Factor (NGF) Vascular Endothelial Growth Factor Platelet Derived Growth Factor A, Insulin like Growth Factor (IGF-I), Epidermal Growth Factor (EGF), Fibroblast Growth Factor (FGF) basic and acidic, Hepatocyte Growth Factor (HGF), Tyrosine Hydroxylase (TH), Aromatic amino acid Decarboxylase (AADC), GTP cyclohydrolase I (GTPCHI), Tryptophan Hydroxylation (Tph) and Insulin identified in Table I will be targeted to modulate expression of neurotrophic factors using genome editing tools.

88. In another embodiment the genome editing reagents specifically gRNAs identified in described in claim 12 can be combined in different premutation to generate most effective treatment and can be delivered using mRNA/LNP complex, AAV or lentiviruses.

89. Method of diagnosis of severe mental health disease based on detection of the following SNPs.
rs4851921, rs9310709, rs53576, rs6449197, rs6295, rs71557378, rs62474683, rs745527286, rs10994330, rs1938526, rs6265, rs2298526, rs242939, rs242941, rs4309482, rs12966547, rs4680

90. Method of treatment of severe mental health diseases identified by SNPs outlined in claim 2 and any other SNPs discovered to be positively correlated with severe mental health diseases

91. Preferred mental health conditions covered by this method patent include but not limited to Suicide, suicidal behavior, severe depression, sever anxiety, obsessive compulsive disorder (OCD), schizophrenia, bipolar disorder and borderline disorders, severe anger, and inability to cope with stress.

92. Method of treating learning disability (LD) gene associated with severe mental health using the correct copy of the normal gene which is affected in severe LD for example Rad6 (Ubiquitin-conjugating enzyme E2).

93. The method described here can be used to change not only the SNP included in this embodiment but also it could be used to change expression of gene as determined by mRNA and protein expression by targeting specific regions in genome that affect chromosomal structure, packing folding and unfolding, region located upstream of critical genes that influence expression of gene mentioned in this embodiment such as those located in promoter region of critical genes. Silencing and activation gene expression can be achieved in a variety of ways through genome editing targeting specific sequences in the promoter region. In some cases RNA editing technologies can also be used to introduce changes into mRNA or siRNA to develop therapies that could affect stability of RNA this leading to increase or decrease in protein expression of certain regulators of brain biology.
